(19) United States
(12) Patent Application Publication
(10) Pub. No.: US 2006/0172932 A1
(43) Pub. Date: Aug. 3, 2006

(54) Novel erythropoietin receptor agonists

(76) Inventors: Neena L. Summers, St. Charles, MO (US); Charles A. McWherter, Wildwood, MO (US); Yiqing Feng, St. Louis, MO (US)

Correspondence Address: Winstead Sechrest & Minick P.C., P.O. Box 50784, Dallas, TX, 75201, US

(21) Appl. No.: 10/972,962

(22) Filed: Oct. 25, 2004

Related U.S. Application Data
(63) Continuation of application No. 08/954,954, filed on Oct. 21, 1997, now abandoned.
(60) Provisional application No. 60/034,044, filed on Oct. 25, 1996.

(51) Int. Cl. A61K 38/18; C07K 14/505

(52) U.S. Cl. 514/12; 530/399

(57) ABSTRACT

Disclosed are novel Erythropoietin receptor agonist proteins, DNAs which encode the Erythropoietin receptor agonist proteins, methods of making the Erythropoietin receptor agonist proteins and methods of using the Erythropoietin receptor agonist proteins.

### CROSS-REFERENCES TO RELATED APPLICATIONS

The present application claims priority is a continuation of U.S. patent application Ser. No. 08/954,954, filed Oct. 21, 1997, which claims priority under Title 35, United States Code, §119 of U.S. Provisional application Serial No. 60/034,044, filed Oct. 25, 1996.

### FIELD OF THE INVENTION

The present invention relates to human Erythropoietin (EPO) receptor agonists. These EPO receptor agonists retain one or more activities of native EPO and may also show improved hematopoietic cell-stimulating activity and/or an improved activity profile which may include reduction of undesirable biological activities associated with native EPO and/or have improved physical properties which may include increased solubility, stability and refold efficiency.

### BACKGROUND OF THE INVENTION

Colony stimulating factors which stimulate the differentiation and/or proliferation of bone marrow cells have generated much interest because of their therapeutic potential for restoring depressed levels of hematopoietic stem cell-derived cells.

Erythropoietin is a naturally-occurring glycoprotein hormone with a molecular weight that was first reported to be approximately 39,000 daltons (T. Miyaki et al., J. Biol. Chem. 252:5558-5564 (1977)). The mature hormone is 166 amino acids long and the “prepro” form of the hormone, with its leader peptide, is 193 amino acids long (F. Lin, U.S. Pat. No. 4,703,008). The mature hormone has a molecular weight, calculated from its amino acid sequence, of 18,399 daltons (K. Jacobs et al., Nature 313:806-810 (1985); J. K. Browne et al., Cold Spring Harbor Symp. Quant. Biol. 5:1693-702 (1986).

The first mutant erythropoietins (i.e., erythropoietin analogs), prepared by making amino acid substitutions and deletions, have demonstrated reduced or unimproved activity. As described in U.S. Pat. No. 4,703,008, replacement of the tyrosine residues at positions 15, 40 and 145 with phenylalanine residues, replacement of the cysteine residue at position 7 with an histidine, substitution of the proline at position 2 with an asparagine, deletion of residues 2-6, deletion of residues 163-166, and deletion of residues 27-55 does not result in an apparent increase in biological activity. The Cys⁷-to-His⁷ mutation eliminates biological activity. A series of mutant erythropoietins with a single amino acid substitution at asparagine residues 24, 38 or 83 show severely reduced activity (substitution at position 24) or exhibit rapid intracellular degradation and apparent lack of secretion (substitution at residue 38 or 183). Elimination of the O-linked glycosylation site at serine126 results in rapid degradation or lack of secretion of the erythropoietin analog (S. Dube et al., J. Biol. Chem. 33:17516-17521 (1988). These authors conclude that glycosylation sites at residues 38, 83 and 126 are required for proper secretion and that glycosylation sites located at residues 24 and 38 may be involved in the biological activity of mature erythropoietin.

Deglycosylated erythropoietin is fully active in in vitro bioassays (M. S. Dorsdal et al., Endocrinology 116:2293-2299 (1985); U.S. Pat. No. 4,703,008; E. Tsuda et al., Eur J. Biochem. 266:20434-20439 (1991). However, glycosylation of erythropoietin is widely accepted to play a critical role in the in vivo activity of the hormone (P. H. Lowy et al., Nature 185:102-105 (1960); E. Goldwasser and C. K. H. Kung, Ann. N.Y. Acad. Science 149:49-53 (1968); W. A. Lukowsky and R. H. Painter, Can. J. Biochem. :909-917 (1972); D. W. Briggs et al., Amer. J. Phys. 201:1385-1388 (1974); J. C. Schooley, Exp. Hematol. 13:994-998; N. Imai et al., Eur. J. Biochem. 194:457-462 (1990); M. S. Dordal et al., Endocrinology 116:2293-2299 (1985); E. Tsuda et al., Eur. J. Biochem. 188:405-411 (1990); U.S. Pat. No. 4,703,008; J. K. Brown et al., Cold Spring Harbor Symposia on Quant. Biol. 51:693-702 (1986); and K. Yamaguchi et al., J. Biol. Chem. 266:20434-20439 (1991). The lack if in vivo biological activity of deglycosylated analogs of erythropoietin is attributed to a rapid clearance of the deglycosylated hormone from the circulation of treated animals. This view is supported by direct comparison of the plasma half-life of glycosylated and deglycosylated erythropoietin (J. C. Spivak and B. B. Hoyans, Blood 73:90-99 (1989), and M. N. Fukuda, et al., Blood 73:84-89 (1989).

Oligonucleotide-directed mutagenesis of erythropoietin glycosylation sites has effectively probed the function of glycosylation but has failed, as yet, to provide insight into an effective strategy for significantly improving the characteristics of the hormone for therapeutic applications.

A series of single amino acid substitution or deletion mutants have been constructed, involving amino acid residues 15, 24, 49, 76, 78, 83, 143, 145, 160, 162, 163, 164, 165 and 166. In these mutants are altered the carboxy terminus, the glycosylation sites, and the tyrosine residues of erythropoietin. The mutants have been administered to animals while monitoring hemoglobin, hematocrit and reticulocyte levels (EP No. 0 409 113). While many of these mutants retain in vivo biological activity, none show a significant increase in their ability to raise hemoglobin, hematocrit or reticulocyte (the immediate precursor of an erythrocyte) levels when compared to native erythropoietin.

Another set of mutants has been constructed to probe the function of residues 99-119 (domain 1) and residues 111-129 (domain 2) (Y. Chern et al., Eur. J. Biochem. 202:225-230 (1991)). The domain 1 mutants are rapidly degraded and inactive in an in vitro bioassay while the domain 2 mutants, at best, retain in vitro activity. These mutants also show no enhanced in vivo biological activity as compared to wild-type, human erythropoietin. These authors conclude that residues 99-119 play a critical role in the structure of erythropoietin.

The human erythropoietin molecule contains two disulfide bridges, one linking the cysteine residues at positions 7 and 161, and a second connecting cysteines at positions 29 and 33 (P.H. Lai et al., J. Biol. Chem. 261:3116-3121 (1986)). Oligonucleotide-directed mutagenesis has been used to probe the function of the disulfide bridge linking cysteines 29 and 33 in human erythropoietin. The cysteine at position 33 has been converted to a proline residue, which, mimics the structure of murine erythropoietin at this residue. The resulting mutant has greatly reduced in vitro activity. The loss of activity is so severe that the authors conclude that the disulfide bridge between residues 29 and 33 is essential for erythropoietin function (F. K. Lin, Molecular and Cellular Aspects of Erythropoietin and Erythropoiesis, pp. 23-36, ed. I. N. Rich, Springer-Verlag, Berlin (1987)).

U.S. Pat. No. 4,703,008 by Lin, F-K. (hereinafter referred to as “the '008 patent”) speculates about a wide variety of modifications of EPO, including addition, deletion, and substitution analogs of EPO. The '008 patent does not indicate that any of the suggested modifications would increase biological activity per se, although it is stated that deletion of glycosylation sites might increase the activity of EPO produced in yeast (See the '008 patent at column 37, lines 25-28). Also, the '008 patent speculates that EPO analogs which have one or more tyrosine residues replaced with phenylalanine may exhibit an increased or decreased receptor binding affinity.

Australian Patent Application No. AU-A-59145/90 by Fibi, M et al. also discusses a number of modified EPO proteins (EPO muteins). It is generally speculated that the alteration of amino acids 10-55, 70-85, and 130-166 of EPO. In particular, additions of positively charged basic amino acids in the carboxyl terminal region are purported to increase the biological activity of EPO.

U.S. Pat. No. 4,835,260 by Shoemaker, C. B. discusses modified EPO proteins with amino acid substitutions of the methionine at position 54 and asparagine at position 38. Such EPO muteins are thought to have improved stability but are not proposed to exhibit any increase in biological activity relative to wild type EPO.

WO 91/05867 discloses analogs of human erythropoietin having a greater number of sites for carbohydrate attachment than human erythropoietin, such as EPO (Asn⁶⁹), EPO (Asn¹²⁵, Ser¹²⁷), EPO (Thr¹²⁵), and EPO (Pro¹²⁴, Thr¹²⁵)

WO 94 /24160 discloses erythropoietin muteins which have enhanced activity, specifically amino acid substitutions at positions 20, 49, 73, 140, 143, 146, 147 and 154.

WO 94/25055 discloses erythropoietin analogs, including EPO (X³³, Cys¹³⁹ , des-Arg¹⁶⁶) and EPO (Cys¹³⁹, des-Arg ¹⁶⁶)

### Rearrangement of Protein Sequences

In evolution, rearrangements of DNA sequences serve an important role in generating a diversity of protein structure and function. Gene duplication and exon shuffling provide an important mechanism to rapidly generate diversity and thereby provide organisms with a competitive advantage, especially since the basal mutation rate is low (Doolittle, Protein Science 1:191-200, 1992).

The development of recombinant DNA methods has made it possible to study the effects of sequence transposition on protein folding, structure and function. The approach used in creating new sequences resembles that of naturally occurring pairs of proteins that are related by linear reorganization of their amino acid sequences (Cunningham, et al., Proc. Natl. Acad. Sci. U.S.A. 76:3218-3222, 1979; Teather & Erfle, J. Bacteriol. 172: 3837-3841, 1990; Schimming et al., Eur. J. Biochem. 204: 13-19, 1992; Yamiuchi and Minamikawa, FEBS Lett. 260:127-130, 1991: MacGregor et al., FEBS Lett. 378:263-266, 1996). The first in vitro application of this type of rearrangement to proteins was described by Goldenberg and Creighton (J. Mol. Biol. 165:407-413, 1983). A new N-terminus is selected at an internal site (breakpoint) of the original sequence, the new sequence having the same order of amino acids as the original from the breakpoint until it reaches an amino acid that is at or near the original C-terminus. At this point the new sequence is joined, either directly or through an additional portion of sequence (linker), to an amino acid that is at or near the original N-terminus, and the new sequence continues with the same sequence as the original until it reaches a point that is at or near the amino acid that was N-terminal to the breakpoint site of the original sequence, this residue forming the new C-terminus of the chain.

This approach has been applied to proteins which range in size from 58 to 462 amino acids (Goldenberg & Creighton, J. Mol. Biol. 165:407-413, 1983; Li & Coffino, Mol. Cell. Biol. 13:2377-2383, 1993). The proteins examined have represented a broad range of structural classes, including proteins that contain predominantly α-helix (interleukin-4; Kreitman et al., Cytokine 7:311-318, 1995), β-sheet (interleukin-1; Horlick et al., Protein Eng. 5:427-431, 1992), or mixtures of the two (yeast phosphoribosyl anthranilate isomerase; Luger et al., Science 243:206-210, 1989). Broad categories of protein function are represented in these sequence reorganization studies:

Enzymes

| | |
|---|---|
| T4 lysozyme | Zhang et al., Biochemistry 32: 12311-12318 |
| | (1993); Zhang et al., Nature Struct. Biol. |
| | 1: 434-438 (1995) |
| dihydrofolate | Buchwalder et al., Biochemistry 31: 1621-1630 |
| | (1994); Protasova et al., Prot. Eng. |
| | 7: 1373-1377 (1995) |
| ribonuclease T1 | Mullins et al., J. Am. Chem. Soc. 116: |
| | 5529-5533 (1994); Garrett et al., Protein |
| | Science 5: 204-211 (1996) |
| Bacillus β-glucanse | Hahn et al., Proc. Natl. Acad. Sci. U.S.A. |
| | 91: 10417-10421 (1994) |
| aspartate | Yang & Schachman, Proc. Natl. Acad. Sci. |
| transcarbamoylase | U.S.A. 90: 11980-11984 (1993) |
| phosphoribosyl | Luger et al., Science 243: 206-210 (1989); |
| anthranilate | Luger et al., Prot. Eng. 3: 249-258 (1990) |
| isomerase | |
| pepsin/pepsinogen | Lin et al., Protein Science 4: 159-166 |
| | (1995) |
| glyceraldehyde-3- | Vignais et al., Protein Science 4: 994-1000 |
| phosphate dehydro- | (1995) |
| genase | |
| ornithine | Li & Coffino, Mol. Cell. Biol. 13: 2377-2383 |
| decarboxylase | (1993) |
| yeast | Ritco-Vonsovici et al., Biochemistry |
| phosphoglycerate | 34: 16543-16551 (1995) |
| dehydrogenase | |

Enzyme Inhibitor

| | |
|---|---|
| basic pancreatic | Goldenberg & Creighton, J. Mol. Biol. |
| trypsin inhibitor | 165: 407-413 (1983) |

Cytokines

| | |
|---|---|
| interleukin-1β | Horlick et al., Protein Eng. 5: 427-431 |
| | (1992) |
| interleukin-4 | Kreitman et al., Cytokine 7: 311-318 (1995) |

Tyrosine Kinase Recognition Domain

| | |
|---|---|
| α-spectrin SH3 | Viguera, et al., J. Mol. Biol. 247: 670-681 |
| domain | (1995) |

Transmembrane Protein

| | |
|---|---|
| omp A | Koebnik & Krämer, J. Mol. Biol. 250: 617-626 |
| | (1995) |

Chimeric Protein

| | |
|---|---|
| interleukin-4- | Kreitman et al., Proc. Natl. Acad. |
| Pseudomonas exotoxin | Sci. U.S.A. 91: 6889-6893 (1994). |
| fusion molecule | |

The results of these studies have been highly variable. In many cases substantially lower activity, solubility or thermodynamic stability were observed (E. coli dihydrofolate reductase, aspartate transcarbamoylase, phosphoribosyl anthranilate isomerase, glyceraldehyde-3-phosphate dehydrogenase, ornithine decarboxylase, omp A, yeast phosphoglycerate dehydrogenase) . In other cases, the sequence rearranged protein appeared to have many nearly identical properties as its natural counterpart (basic pancreatic trypsin inhibitor, T4 lysozyme, ribonuclease T1, Bacillus β-glucanase, interleukin-1β, α-spectrin SH3 domain, pepsinogen, interleukin-4). In exceptional cases, an unexpected improvement over some properties of the natural sequence was observed, e.g., the solubility and refolding rate for rearranged x-spectrin SH3 domain sequences, and the receptor affinity and anti-tumor activity of transposed interleukin-4-Pseudomonas exotoxin fusion molecule (Kreitman et al., Proc. Natl. Acad. Sci. U.S.A. 91:6889-6893, 1994; Kreitman et al., Cancer Res. 55:3357-3363, 1995).

The primary motivation for these types of studies has been to study the role of short-range and long-range interactions in protein folding and stability. Sequence rearrangements of this type convert a subset of interactions that are long-range in the original sequence into short-range interactions in the new sequence, and vice versa. The fact that many of these sequence rearrangements are able to attain a conformation with at least some activity is persuasive evidence that protein folding occurs by multiple folding pathways (Viguera, et al., J. Mol. Biol. 247:670-681, 1995). In the case of the SH3 domain of α-spectrin, choosing new termini at locations that corresponded to β-hairpin turns resulted in proteins with slightly less stability, but which were nevertheless able to fold.

The positions of the internal breakpoints used in the studies cited here are found exclusively on the surface of proteins, and are distributed throughout the linear sequence without any obvious bias towards the ends or the middle (the variation in the relative distance from the original N-terminus to the breakpoint is ca. 10 to 80% of the total sequence length). The linkers connecting the original N- and C-termini in these studies have ranged from 0 to 9 residues. In one case (Yang & Schachman, Proc. Natl. Acad. Sci. U.S.A. 90:11980-11984, 1993), a portion of sequence has been deleted from the original C-terminal segment, and the connection made from the truncated C-terminus to the original N-terminus. Flexible hydrophilic residues such as Gly and Ser are frequently used in the linkers. Viguera, et al. (J. Mol. Biol. 247:670-681, 1995) compared joining the original N- and C- termini with 3- or 4-residue linkers; the 3-residue linker was less thermodynamically stable. Protasova et al. (Protein Eng. 7:1373-1377, 1994) used 3- or 5-residue linkers in connecting the original N-termini of E. coli dihydrofolate reductase; only the 3-residue linker produced protein in good yield.

### SUMMARY OF THE INVENTION

The modified human EPO receptor agonists of the present invention can be represented by the Formula:
X¹−(L)ₐ−X²
wherein
a is 0 or 1;
X¹ is a peptide comprising an amino acid sequence corresponding to the sequence of residues n+1 through J;
X² is a peptide comprising an amino acid sequence corresponding to the sequence of residues 1 through n;
n is an integer ranging from 1 to J-1; and
L is a linker.

In the formula above the constituent amino acids residues of human EPO are numbered sequentially 1 through J from the amino to the carboxyl terminus. A pair of adjacent amino acids within this protein may be numbered n and n+1 respectively where n is an integer ranging from 1 to J-1. The residue n+1 becomes the new N-terminus of the new EPO receptor agonist and the residue n becomes the new C-terminus of the new EPO receptor agonist.

The present invention relates to novel EPO receptor agonists polypeptides comprising a modified EPO amino acid sequence of the following formula:

```
AlaProProArgLeuIleCysAspSerArgValLeuGluArgTyrLeuLeuGluAlaLys
                                                10 20
GluAlaGluAsnIleThrThrGlyCysAlaGluHisCysSerLeuAsnGluAsnIleThr
                                                30 40
ValProAspThrLysValAsnPheTyrAlaTrpLysArgMetGluValGlyGlnGlnAla
                                                50 60
ValGluValTrpGlnGlyLeuAlaLeuLeuSerGluAlaValLeuArgGlyGlnAlaLeu
                                                70 80
LeuValAsnSerSerGlnProTrpGluProLeuGlnLeuHisValAspLysAlaValSer
                                                90 100
GlyLeuArgSerLeuThrThrLeuLeuArgAlaLeuGlyAlaGlnLysGluAlaIleSer
                                                110 120
ProProAspAlaAlaSerAlaAlaProLeuArgThrIleThrAlaAspThrPheArgLys
                                                130 140
LeuPheArgValTyrSerAsnPheLeuArgGlyLysLeuLysLeuTyrThrGlyGluAla
                                                150 160
CysArgThrGlyAspArg
                        166
```

wherein optionally 1-6 amino acids from the N-terminus and 1-5 from the C-terminus can be deleted from said EPO receptor agonists polypeptide; wherein the N-terminus is joined to the C-terminus directly or through a linker capable of joining the N-terminus to the C-terminus and having new C- and N-termini at amino acids;

23-24
24-25
25-26
26-27
27-28
28-29
29-30
30-31
31-32
32-33
33-34
34-35
35-36
36-37
37-38
38-39
40-41
41-42
43-44
44-45
45-46
46-47
47-48
48-49
50-51
51-52
52-53
53-54
54-55
55-56
56-57
57-58
77-78
78-79
79-80
80-81
81-82
82-83
84-85
85-86
86-87
87-88
88-89
108-109
109-110
110-111
111-112
112-113
113-114
114-115
115-116
116-117
117-118
118-119
119-120
120-121
121-122
122-123
123-124
124-125
125-126
126-127
127-128
128-129
129-130
131-132
respectively; and

said EPO receptor agonist polypeptide may optionally be immediately preceded by (methionine⁻¹), (alanine⁻¹) or (methionine⁻², alanine⁻¹).

The more preferred breakpoints at which new C-terminus and N-terminus can be made are; 23-24, 24-25, 25-26, 27-28, 28-10 29, 29-30, 30-31, 31-32, 32-33, 33-34, 34-35, 35-36, 36-37, 37-38, 38-39, 40-41, 41-42, 42-43, 52-53, 53-54, 54-55, 55-56, 77-78, 78-79, 79-80, 80-81, 81-82, 82-83, 83-84, 84-85, 85-86, 86-87, 87-88, 88-89, 109-110, 110-111, 111-112, 112-113, 113-114, 114-115, 115-116, 116-117, 117-118, 118-119, 119-120, 120-121, 121-122, 122-123, 123-124, 124-125, 125-126, 126-127, 127-128, 128-129, 129-130, 130-131, and 131-132.

The most preferred breakpoints at which new C-terminus and N-terminus can be made are; 23-24, 24-25, 31-32, 32-33, 37-38, 38-39, 82-83, 83-84,85-86, 86-87, 87-88, 125-126, 126-127, and 131-132.

The most preferred breakpoints include glycosylationn sites, non-nuetralizing antibodies, proteolyte cleavage sites.

The EPO receptor agonists of the present invention may contain amino acid substitutions, such as those disclosed in WO 94/24160 or one or more of the glycosylation sites at Asn²⁴, Asn⁸³, and Asn¹²⁶ are changed to other amino acids such as but not limited to Asp or Glu, deletions and/or insertions. It is also intended that the EPO receptor agonists of the present invention may also have amino acid deletions at either/or both the N- and C-termini of the original protein and or deletions from the new N- and/or C-termini of the sequence rearranged proteins in the formulas shown above.

A preferred embodiment of the present invention the linker (L) joining the N-terminus to the C-terminus is a polypeptide selected from the group consisting of:

| | |
|---|---|
| GlyGlyGlySer; | SEQ ID NO:123 |
| GlyGlyGlySerGlyGlyGlySer; | SEQ ID NO:124 |
| GlyGlyGlySerGlyGlyGlySerGlyGlyGlySer; | SEQ ID NO:125 |
| SerGlyGlySerGlyGlySer; | SEQ ID NO:126 |
| GluPheGlyAsnMet; | SEQ ID NO:127 |
| GluPheGlyGlyAsnMet; | SEQ ID NO:128 |
| GluPheGlyGlyAsnGlyGlyAsnMet; | SEQ ID NO:129 |
| and | |
| GlyGlySerAspMetAlaGly. | SEQ ID NO:130 |

The present invention also encompasses recombinant human EPO receptor agonists co-administered or sequentially with one or more additional colony stimulating factors (CSF) including, cytokines, lymphokines, interleukins, hematopoietic growth factors which include but are not limited to GM-CSF, G-CSF, c-mpl ligand (also known as TPO or MGDF), M-CSF, IL-1, IL-4, IL-2, IL-3, IL-5, IL 6, IL-7, IL-8, IL-9, IL-10, IL-11, IL-12, IL-13, IL-15, LIF, human growth hormone, B-cell growth factor, B-cell differentiation factor, eosinophil differentiation factor and stem cell factor (SCF) also known as steel factor or c-kit ligand (herein collectively referred to as “factors”) . These co-administered mixtures may be characterized by having the usual activity of both of the peptides or the mixture may be further characterized by having a biological or physiological activity greater than simply the additive function of the presence of the EPO receptor agonists or the second colony stimulating factor alone. The co-administration may also provide an enhanced effect on the activity or an activity different from that expected by the presence of the EPO or the second colony stimulating factor. The co-administration may also have an improved activity profile which may include reduction of undesirable biological activities associated with native human EPO. In addition to the list above, IL-3 variants taught in WO 94/12639 and WO 94/12638 fusion protein taught in WO 95/21197, and WO 95/21254 G-CSF receptor agonists disclosed in WO 97/12977, c-mpl receptor agonists disclosed in WO 97/12978, IL-3 receptor agonists disclosed in WO 97/12979 and multi-functional receptor agonists taught in WO 97/12985 can be co-administered with the polypeptides of the present invention. As used herein “IL-3 variants” refer to IL-3 variants taught in WO 94/12639 and WO 94/12638. As used herein “fusion proteins” refer to fusion protein taught in WO 95/21197, and WO 95/21254. As used herein “G-CSF receptor agonists” refer to G-CSF receptor agonists disclosed in WO 97/12978. As used herein “c-mpl receptor agonists” refer to c-mpl receptor agonists disclosed in WO 97/12978. As used herein “IL-3 receptor agonists” refer to IL-3 receptor agonists disclosed in WO 97/12979. As used herein “multi-functional receptor agonists” refer to multi-functional receptor agonists taught in WO 97/12985.

In addition, it is envisioned that in vitro uses would include the ability to stimulate bone marrow and blood cell activation and growth before the expanded cells are infused into patients.

It is also envisioned that uses of EPO receptor agonists of the present invention would include blood banking applications, where the EPO receptor agonists are given to a patent to increase the number of red blood cells and blood products removed from the patient, prior to some medical procedure, and the blood products stored and transfused back into the patient after the medical procedure. Additionally, it is envisioned that uses of EPO receptor agonists would include giving the EPO receptor agonists to a blood donor prior to blood donation to increase the number of red blood cells, thereby allowing the donor to safely give more blood.

### BRIEF DESCRIPTION OF THE FIGURES

FIG. 1 schematically illustrates the sequence rearrangement of a protein. The N-terminus (N) and the C-terminus (C) of the native protein are joined through a linker, or joined directly. The protein is opened at a breakpoint creating a new N-terminus (new N) and a new C-terminus (new-C) resulting in a protein with a new linear amino acid sequence. A rearranged molecule may be synthesized de novo as linear molecule and not go through the steps of joining the original N-terminus and the C-terminus and opening of the protein at the breakpoint.

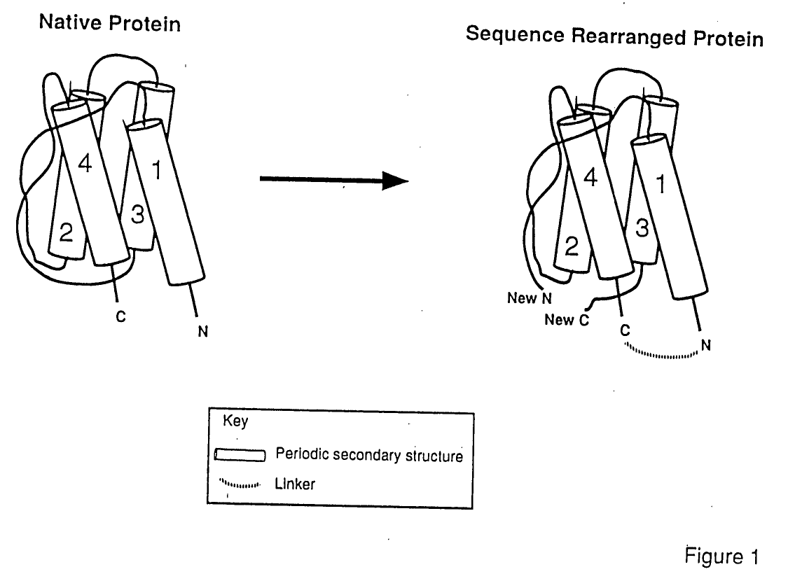

FIG. 2 shows a schematic of Method I, for creating new proteins in which the original N-terminus and C-terminus of the native protein are joined with a linker and different N-terminus and C-terminus of the protein are created. In the example shown the sequence rearrangement results in a new gene encoding a protein with a new N-terminus created at amino acid 97 of the original protein, the original C-terminus (a.a. 174) joined to the amino acid 11 (a.a. 1-10 are deleted) through a linker region and a new C-terminus created at amino acid 96 of the original sequence.

FIG. 3 shows a schematic of Method II, for creating new proteins in which the original N-terminus and C-terminus of the native protein are joined without a linker and different N-terminus and C-terminus of the protein are created. In the example shown the sequence rearrangement results in a new gene encoding a protein with a new N-terminus created at amino acid 97 of the original protein, the original C-terminus (a.a. 174) joined to the original N-terminus and a new C-terminus created at amino acid 96 of the original sequence.

FIG. 4 shows a schematic of Method III, for creating new proteins in which the original N-terminus and C-terminus of the native protein are joined with a linker and different N-terminus and C-terminus of the protein are created. In the example shown the sequence rearrangement results in a new gene encoding a protein with a new N-terminus created at amino acid 97 of the original protein, the original C-terminus (a.a. 174) joined to amino acid 1 through a linker region and a new C-terminus created at amino acid 96 of the original sequence.

FIG. 5 shows a DNA sequence encoding human mature EPO based on the sequence of Lin et al. (PNAS 82:7580-7584, 1985).

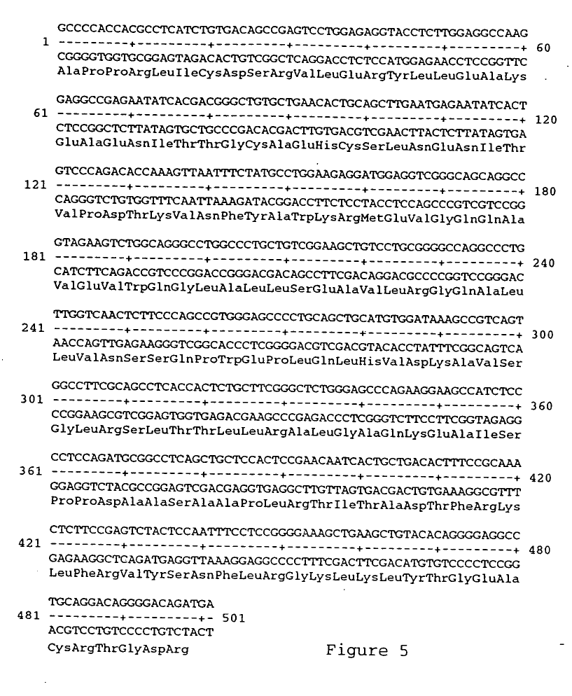

### DETAILED DESCRIPTION OF THE INVENTION

Receptor agonists of the present invention may be useful in the treatment of diseases characterized by decreased levels of red blood cells of the hematopoietic system.

A EPO receptor agonist may be useful in the treatment or prevention of anemia. Many drugs may cause bone marrow suppression or hematopoietic deficiencies. Examples of such drugs are AZT, DDI, alkylating agents and anti-metabolites used in chemotherapy, antibiotics such as chloramphenicol, penicillin, gancyclovir, daunomycin and sulfa drugs, phenothiazones, tranquilizers such as meprobamate, analgesics such as aminopyrine and dipyrone, anti-convulsants such as phenytoin or carbamazepine, antithyroids such as propylthiouracil and methimazole and diuretics. EPO receptor agonists may be useful in preventing or treating the bone marrow suppression or hematopoietic deficiencies which often occur in patients treated with these drugs.

Hematopoietic deficiencies may also occur as a result of viral, microbial or parasitic infections and as a result of treatment for renal disease or renal failure, e.g., dialysis. The present peptide may be useful in treating such hematopoietic deficiency.

Another aspect of the present invention provides plasmid DNA vectors for use in the method of expression of these novel EPO receptor agonists. These vectors contain the novel DNA sequences described above which code for the novel polypeptides of the invention. Appropriate vectors which can transform host cells capable of expressing the EPO receptor agonists include expression vectors comprising nucleotide sequences coding for the EPO receptor agonists joined to transcriptional and translational regulatory sequences which are selected according to the host cells used. Vectors incorporating modified sequences as described above are included in the present invention and are useful in the production of the modified EPO receptor agonist polypeptides. The vector employed in the method also contains selected regulatory sequences in operative association with the DNA coding sequences of the invention and capable of directing the replication and expression thereof in selected host cells.

As another aspect of the present invention, there is provided a method for producing the novel family of human EPO receptor agonists. The method of the present invention involves culturing suitable cells or cell line, which has been transformed with a vector containing a DNA sequence coding for expression of the novel EPO receptor agonist polypeptide. Suitable cells or cell lines may include various strains of bacteria such as E. coli, yeast, mammalian cells, or insect cells may be utilized as host cells in the method of the present invention.

Other aspects of the present invention are methods and therapeutic compositions for treating the conditions referred to above. Such compositions comprise a therapeutically effective amount of one or more of the EPO receptor agonists of the present invention in a mixture with a pharmaceutically acceptable carrier. This composition can be administered either parenterally, intravenously or subcutaneously. When administered, the therapeutic composition for use in this invention is preferably in the form of a pyrogen-free, parenterally acceptable aqueous solution. The preparation of such a parenterally acceptable protein solution, having due regard to pH, isotonicity, stability and the like, is within the skill of the art.

Administration will be in accordance with a dosage regimen that will be readily ascertained by the skilled, based on in vivo specific activity of the analog in comparison with human erythropoietin and based on what is now known in the art concerning the administration of human erythropoietin for inducing erythropoiesis and treating various conditions, such as anemia, in humans, including anemia in patients suffering from renal failure. Dosage of an analog of the invention may vary somewhat from individual to individual, depending on the particular analog and its specific in vivo activity, the route of administration, the medical condition, age, weight or sex of the patient, the patient's sensitivities to the analog or components of vehicle, and other factors which the attending physician will be capable of readily taking into account. With regard to therapeutic uses of analogs of the invention, reference is made to U.S. Pat. Nos. 4,703,008 and 4,835,260; see also the chapter on (recombinant) [des-Arg¹⁶⁶] human erythropoietin at pages 591-595 of the Physicians' Desk Commercially available preparations of recombinant [des-Arg¹⁶⁶] human erythropoietin have 2,000, 3,000, 4,000 or 10,000 units of the glycohormone per mL in preservative-free aqueous solution with 2.5 mg/mL human serum albumin, 5.8 mg/mL sodium citrate, 5.8 mg/mL NaCl, and 0.06 mg/mL citric acid, pH 6.9 (+/−0.3).

Recombinantly produced EPO has proven especially useful for the treatment of patients suffering from impaired red blood cell production (Physicians Desk Reference (PDR) . 1993 edition, pp 602-605). Recombinant EPO has proven effective in treating anemia associated with chronic renal failure and HIV-Infected individuals suffering from lowered endogenous EPO levels related to therapy with Zidovudine (AZT) (See PDR, 1993 edition, at page 6002).

Modifications of the EPO protein which would improve its utility as a tool for diagnosis or treatment of blood disorders are certainly desirable. In particular, modified forms of EPO exhibiting enhanced biological activity would be more effective and efficient than native EPO in the therapy setting when it is necessary to administer EPO to the patient, enabling administration less frequently and/or at a lower dose. Administration of reduced amounts of EPO would also presumably reduce the risk of adverse effects associated with EPO treatment, such as hypertension, seizures, headaches, etc. (See PDR, 1993 edition, at pp. 603-604). The EPO receptor agonists of the present invention may also have improved stability and hence increased half-life which would allow for the production of a non-glycosylated form of EPO in a bacterial expression system at a much lower cost. Due it's increased half-life this non-glycosylated form of EPO would have an increased in vivo activity compared de-glycosylated EPO.

The therapeutic method and compositions may also include co-administration with other hematopoietic factors. A non-exclusive list of other appropriate hematopoietins, colony stimulating factors (CSFs) and interleukins for simultaneous or serial co-administration with the polypeptides of the present invention includes GM-CSF, G-CSF, c-mpl ligand (also known as TPO or MGDF), M-CSF, IL-1, IL-4, IL-2, IL-3, IL-5, IL 6, IL-7, IL-8, IL-9, IL-10, IL-11, IL-12, IL-13, IL-15, LIF, human growth hormone, B-cell growth factor, B-cell differentiation factor, eosinophil differentiation factor and stem cell factor (SCF) also known as steel factor or c-kit ligand (herein collectively referred to as “factors”), or combinations thereof. In addition to the list above, IL-3 variants taught in WO 94/12639 and WO 94/12638 fusion protein taught in WO 95/21197, and WO 95/21254 G-CSF receptor agonists disclosed in WO 97/12977, c-mpl receptor agonists disclosed in WO 97/12978, IL-3 receptor agonists disclosed in WO 97/12979 and multi-functional receptor agonists taught in WO 97/12985 can be co-administered with the polypeptides of the present invention.

The EPO receptor agonists of the present invention may be useful in the mobilization of hematopoietic progenitors and stem cells in peripheral blood. Peripheral blood derived progenitors have been shown to be effective in reconstituting patients in the setting of autologous marrow transplantation.

The EPO receptor agonists of the present invention may also be useful in the ex vivo expansion of hematopoietic progenitors. Colony stimulating factors (CSFs), such as G-CSF, have been administered alone, co-administered with other CSFs, or in combination with bone marrow transplants subsequent to high dose chemotherapy to treat the anemia, neutropenia and thrombocytopenia which are often the result of such treatment.

Another aspect of the invention provides methods of sustaining and/or expanding hematopoietic precursor cells which includes inoculating the cells into a culture vessel which contains a culture medium that has been conditioned by exposure to a stromal cell line such as HS-5 (WO 96/02662, Roecklein and Torok-Strob, Blood 85:997-1105, 1995) that has been supplemented with a EPO receptor agonist of the present invention.

### Determination of the Linker

The length of the amino acid sequence of the linker can be selected empirically or with guidance from structural information, or by using a combination of the two approaches.

When no structural information is available, a small series of linkers can be prepared for testing using a design whose length is varied in order to span a range from 0 to 50 A and whose sequence is chosen in order to be consistent with surface exposure (hydrophilicity, Hopp & Woods, Mol. Immunol. 20: 483-489, 1983; Kyte & Doolittle, J. Mol. Biol. 157:105-132, 1982; solvent exposed surface area, Lee & Richards, J. Mol. Biol. 55:379-400, 1971) and the ability to adopt the necessary conformation without deranging the configuration of the EPO receptor agonist (conformationally flexible; Karplus & Schulz, Naturwissenschaften 72:212-213, (1985). Assuming an average of translation of 2.0 to 3.8 Å per residue, this would mean the length to test would be between 0 to 30 residues, with 0 to 15 residues being the preferred range. Exemplary of such an empirical series would be to construct linkers using a cassette sequence such as Gly-Gly-Gly-Ser repeated n times, where n is 1, 2, 3 or 4. Those skilled in the art will recognize that there are many such sequences that vary in length or composition that can serve as linkers with the primary consideration being that they be neither excessively long nor short (cf., Sandhu, Critical Rev. Biotech. 12: 437-462, 1992); if they are too long, entropy effects will likely destabilize the three-dimensional fold, and may also make folding kinetically impractical, and if they are too short, they will likely destabilize the molecule because of torsional or steric strain.

Those skilled in the analysis of protein structural information will recognize that using the distance between the chain ends, defined as the distance between the c-alpha carbons, can be used to define the length of the sequence to be used, or at least to limit the number of possibilities that must be tested in an empirical selection of linkers. They will also recognize that it is sometimes the case that the positions of the ends of the polypeptide chain are ill-defined in structural models derived from x-ray diffraction or nuclear magnetic resonance spectroscopy data, and that when true, this situation will therefore need to be taken into account in order to properly estimate the length of the linker required. From those residues whose positions are well defined are selected two residues that are close in sequence to the chain ends, and the distance between their c-alpha carbons is used to calculate an approximate length for a linker between them. Using the calculated length as a guide, linkers with a range of number of residues (calculated using 2 to 3.8 Å per residue) are then selected. These linkers may be composed of the original sequence, shortened or lengthened as necessary, and when lengthened the additional residues may be chosen to be flexible and hydrophilic as described above; or optionally the original sequence may be substituted for using a series of linkers, one example being the “Gly-Gly-Gly-Ser” cassette approach mentioned above; or optionally a combination of the original sequence and new sequence having the appropriate total length may be used.

### Determination of the Amino and Carboxyl Termini of EPO Receptor Agonists

Sequences of EPO receptor agonists capable of folding to biologically active states can be prepared by appropriate selection of the beginning (amino terminus) and ending (carboxyl terminus) positions from within the original polypeptide chain while using the linker sequence as described above. Amino and carboxyl termini are selected from within a common stretch of sequence, referred to as a breakpoint region, using the guidelines described below A novel amino acid sequence is thus generated by selecting amino and carboxyl termini from within the same breakpoint region. In many cases the selection of the new termini will be such that the original position of the carboxyl terminus immediately preceded that of the amino terminus. However, those skilled in the art will recognize that selections of termini anywhere within the region may function, and that these will effectively lead to either deletions or additions to the amino or carboxyl portions of the new sequence.

It is a central tenet of molecular biology that the primary amino acid sequence of a protein dictates folding to the three-dimensional structure necessary for expression of its biological function. Methods are known to those skilled in the art to obtain and interpret three-dimensional structural information using x-ray diffraction of single protein crystals or nuclear magnetic resonance spectroscopy of protein solutions. Examples of structural information that are relevant to the identification of breakpoint regions include the location and type of protein secondary structure (alpha and 3-10 helices, parallel and anti-parallel beta sheets, chain reversals and turns, and loops; Kabsch & Sander, Biopolymers 22: 2577-2637, 1983; the degree of solvent exposure of amino acid residues, the extent and type of interactions of residues with one another (Chothia, Ann. Rev. Biochem. 53:537-572; 1984) and the static and dynamic distribution of conformations along the polypeptide chain (Alber & Mathews, Methods Enzymol. 154: 511-533, 1987). In some cases additional information is known about solvent exposure of residues; one example is a site of post-translational attachment of carbohydrate which is necessarily on the surface of the protein. When experimental structural information is not available, or is not feasible to obtain, methods are also available to analyze the primary amino acid sequence in order to make predictions of protein tertiary and secondary structure, solvent accessibility and the occurrence of turns and loops. Biochemical methods are also sometimes applicable for empirically determining surface exposure when direct structural methods are not feasible; for example, using the identification of sites of chain scission following limited proteolysis in order to infer surface exposure (Gentile & Salvatore, Eur. J. Biochem. 218:603-621, 1993)

Thus using either the experimentally derived structural information or predictive methods (e.g., Srinivisan & Rose Proteins: Struct., Funct. & Genetics, 22: 81-99, 1995) the parental amino acid sequence is inspected to classify regions according to whether or not they are integral to the maintenance of secondary and tertiary structure. The occurrence of sequences within regions that are known to be involved in periodic secondary structure (alpha and 3-10 helices, parallel and anti-parallel beta sheets) are regions that should be avoided. Similarly, regions of amino acid sequence that are observed or predicted to have a low degree of solvent exposure are more likely to be part of the so-called hydrophobic core of the protein and should also be avoided for selection of amino and carboxyl termini. In contrast, those regions that are known or predicted to be in surface turns or loops, and especially those regions that are known not to be required for biological activity, are the preferred sites for location of the extremes of the polypeptide chain. Continuous stretches of amino acid sequence that are preferred based on the above criteria are referred to as a breakpoint region.

### Materials and Methods Recombinant DNA methods

Unless noted otherwise, all specialty chemicals were obtained from Sigma Co., (St. Louis, Mo.). Restriction endonucleases and T4 DNA ligase were obtained from New England Biolabs (Beverly, Mass.) or Boehringer Mannheim (Indianapolis, Ind.).

### Transformation of E. coli Strains

E. coli strains, such as DH5α™ (Life Technologies, Gaithersburg, Md.) and TGl (Amersham Corp., Arlington Heights, Ill.) are used for transformation of ligation reactions and are the source of plasmid DNA for transfecting mammalian cells. E. coli strains, such as MON105 and JM101, can be used for expressing the EPO receptor agonist of the present invention in the cytoplasm or periplasmic space.

MON105 ATCC#55204: F-, lamda-,IN(rrnD, rrE)1, rpoD+, rpoH358 DH5α™: F-, phi80dlacZdeltaM15, delta(lacZYA-argF)U169, deoR, recA1, endA1, hsdR17(rk−,mk+), phoA, supE44lamda-, thi-1, gyrA96, relA1

TG1: delta(lac-pro), supE, thi-1, hsdD5/F′ (traD36, proA+B+, lacIq, lacZdeltaM15)

DH5α™ Subcloning efficiency cells are purchased as competent cells and are ready for transformation using the manufacturer's protocol, while both E. coli strains TG1 and MON105 are rendered competent to take up DNA using a CaCl₂ method. Typically, 20 to 50 mL of cells are grown in LB medium (1% Bacto-tryptone, 0.5% Bacto-yeast extract, 150 mM NaCl) to a density of approximately 1.0 optical density unit at 600 nanometers (OD600) as measured by a Baush & Lomb Spectronic spectrophotometer (Rochester, N.Y.) . The cells are collected by centrifugation and resuspended in one-fifth culture volume of CaCl₂ solution (50 mM CaCl₂, 10 mM Tris-Cl, pH7.4) and are held at 4° C. for 30 minutes. The cells are again collected by centrifugation and resuspended in one-tenth culture volume of CaCl₂ solution. Ligated DNA is added to 0.2 mL of these cells, and the samples are held at 4° C. for 1 hour. The samples are shifted to 42° C. for two minutes and 1 mL of LB is added prior to shaking the samples at 37° C. for one hour. Cells from these samples are spread on plates (LB medium plus 1.5% Bacto-agar) containing either ampicillin (100 micrograms/mL, ug/mL) when selecting for ampicillin-resistant transformants, or spectinomycin (75 ug/mL) when selecting for spectinomycin-resistant transformants. The plates are incubated overnight at 37° C. Single colonies are picked, grown in LB supplemented with appropriate antibiotic for 6-16 hours at 37° C. with shaking. Colonies are picked and inoculated into LB plus appropriate antibiotic (100 ug/mL ampicillin or 75 ug/mL spectinomycin) and are grown at 37° C. while shaking. Before harvesting the cultures, 1 ul of cells are analyzed by PCR for the presence of a EPO receptor agonist gene. The PCR is carried out using a combination of primers that anneal to the EPO receptor agonist gene and/or vector. After the PCR is complete, loading dye is added to the sample followed by electrophoresis as described earlier. A gene has been ligated to the vector when a PCR product of the expected size is observed.

### Methods for Creation of Genes with New N-terminus/C-terminus

Method I. Creation of Genes with New N-terminus/C-terminus which Contain a Linker Region.

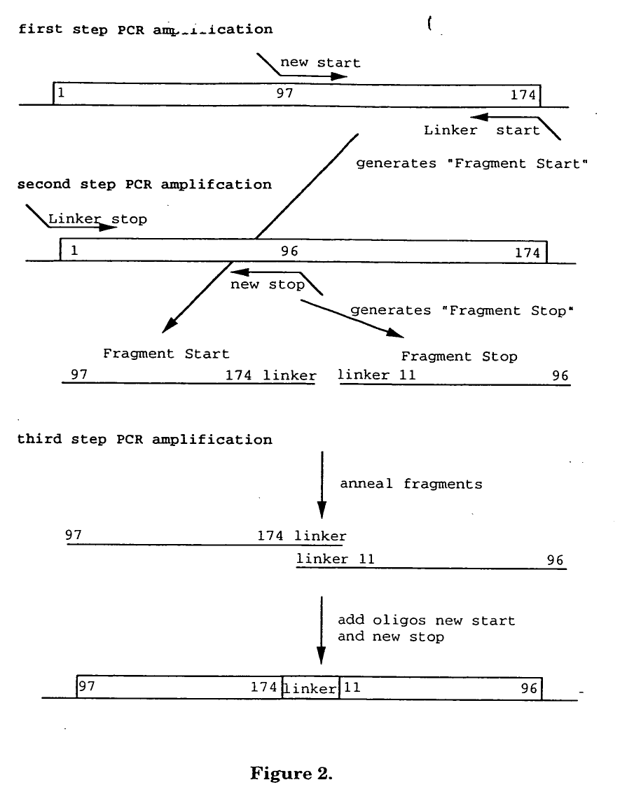

Genes with new N-terminus/C-terminus which contain a linker region separating the original C-terminus and N-terminus can be made essentially following the method described in L. S. Mullins, et al J. Am. Chem. Soc. 116, 5529-5533 (1994). Multiple steps of polymerase chain reaction (PCR) amplifications are used to rearrange the DNA sequence encoding the primary amino acid sequence of the protein. The steps are illustrated in FIG. 2.

In the first step, the primer set (“new start” and “linker start”) is used to create and amplify, from the original gene sequence, the DNA fragment (“Fragment Start”) that contains the sequence encoding the new N-terminal portion of the new protein followed by the linker that connects the C-terminal and N-terminal ends of the original protein. In the second step, the primer set (“new stop” and “linker stop”) is used to create and amplify, from the original gene sequence, the DNA fragment (“Fragment Stop”) that encodes the same linker as used above, followed by the new C-terminal portion of the new protein. The “new start” and “new stop” primers are designed to include the appropriate restriction enzyme recognition sites which allow cloning of the new gene into expression plasmids. Typical PCR conditions are one cycle 95° C. melting for two minutes; 25 cycles 94° C. denaturation for one minute, 50° C. annealing for one minute and 72° C. extension for one minute; plus one cycle 72° C. extension for seven minutes. A Perkin Elmer GeneAmp PCR Core Reagents kit is used. A 100 ul reaction contains 100 pmole of each primer and one ug of template DNA; and 1× PCR buffer, 200 uM dGTP, 200 uM DATP, 200 uM dTTP, 200 uM dCTP, 2.5 units AmpliTaq DNA polymerase and 2 mM MgCl₂. PCR reactions are performed in a Model 480 DNA thermal cycler (Perkin Elmer Corporation, Norwalk, Conn.).

“Fragment Start” and “Fragment Stop”, which have complementary sequence in the linker region and the coding sequence for the two amino acids on both sides of the linker, are joined together in a third PCR step to make the full-length gene encoding the new protein. The DNA fragments “Fragment Start” and “Fragment Stop” are resolved on a 1% TAE gel, stained with ethidium bromide and isolated using a Qiaex Gel Extraction kit (Qiagen). These fragments are combined in equimolar quantities, heated at 70° C. for ten minutes and slow cooled to allow annealing through their shared sequence in “linker start” and “linker stop”. In the third PCR step, primers “new start” and “new stop” are added to the annealed fragments to create and amplify the full-length new N-terminus/C-terminus gene. Typical PCR conditions are one cycle 95° C. melting for two minutes; 25 cycles 94° C. denaturation for one minute, 60° C. annealing for one minute and 72° C. extension for one minute; plus one cycle 72° C. extension for seven minutes. A Perkin Elmer GeneAmp PCR Core Reagents kit is used. A 100 ul reaction contains 100 pmole of each primer and approximately 0.5 ug of DNA; and 1× PCR buffer, 200 uM dGTP, 200 uM DATP, 200 uM dTTP, 200 uM dCTP, 2.5 units AmpliTaq DNA polymerase and 2 mM MgCl₂. PCR reactions are purified using a Wizard PCR Preps kit (Promega).

Method II. Creation of Genes with New N-terminus/C-terminus without a Linker Region.

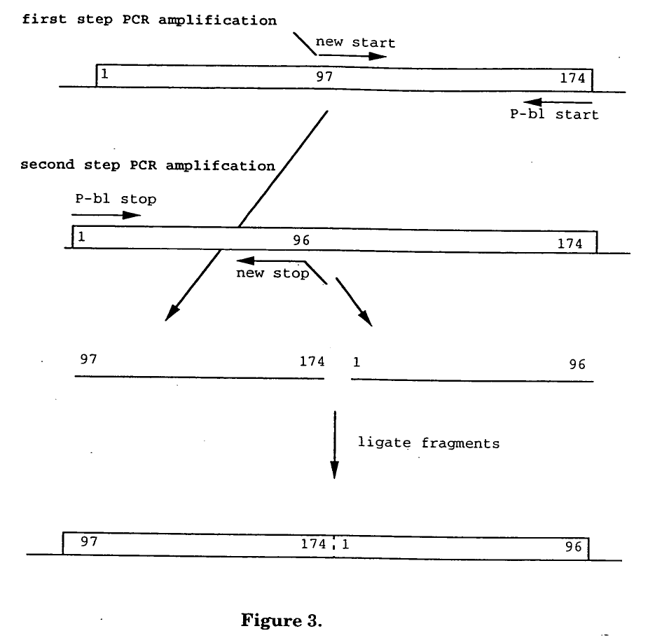

New N-terminus/C-terminus genes without a linker joining the original N-terminus and C-terminus can be made using two steps of PCR amplification and a blunt end ligation. The steps are illustrated in FIG. 3. In the first step, the primer set (“new start” and “P-bl start”) is used to create and amplify, from the original gene sequence, the DNA fragment (“Fragment Start”) that contains the sequence encoding the new N-terminal portion of the new protein. In the second step, the primer set (“new stop” and “P-bl stop”) is used to create and amplify, from the original gene sequence, the DNA fragment (“Fragment Stop”) that contains the sequence encoding the new C-terminal portion of the new protein. The “new start” and “new stop” primers are designed to include appropriate restriction sites which allow cloning of the new gene into expression vectors. Typical PCR conditions are one cycle 95° C. melting for two minutes; 25 cycles 94° C. denaturation for one minute, 50° C. annealing for 45 seconds and 72° C. extension for 45 seconds. Deep Vent polymerase (New England Biolabs) is used to reduce the occurrence of overhangs in conditions recommended by the manufacturer. The “P-bl start” and “P-bl stop” primers are phosphorylated at the 5′ end to aid in the subsequent blunt end ligation of “Fragment Start” and “Fragment Stop” to each other. A 100 ul reaction contained 150 pmole of each primer and one ug of template DNA; and 1× Vent buffer (New England Biolabs), 300 uM dGTP, 300 uM DATP, 300 uM dTTP, 300 uM dCTP, and 1 unit Deep Vent polymerase. PCR reactions are performed in a Model 480 DNA thermal cycler (Perkin Elmer Corporation, Norwalk, Conn.). PCR reaction products are purified using a Wizard PCR Preps kit (Promega).

The primers are designed to include appropriate restriction enzyme recognition sites which allow for the cloning of the new gene into expression vectors. Typically “Fragment Start” is designed to create a NcoI restriction site , and “Fragment Stop” is designed to create a HindIII restriction site. Restriction digest reactions are purified using a Magic DNA Clean-up System kit (Promega). Fragments Start and Stop are resolved on a 1% TAE gel, stained with ethidium bromide and isolated using a Qiaex Gel Extraction kit (Qiagen). These fragments are combined with and annealed to the ends of the ˜3800 base pair NcoI/HindIII vector fragment of pMON3934 by heating at 50° C. for ten minutes and allowed to slow cool. The three fragments are ligated together using T4 DNA ligase (Boehringer Mannheim). The result is a plasmid containing the full-length new N-terminus/C-terminus gene. A portion of the ligation reaction is used to transform E. coli strain DH5α cells (Life Technologies, Gaithersburg, Md.). Plasmid DNA is purified and sequence confirmed as below.

Method III. Creation of New N-terminus/C-terminus Genes by Tandem-duplication Method

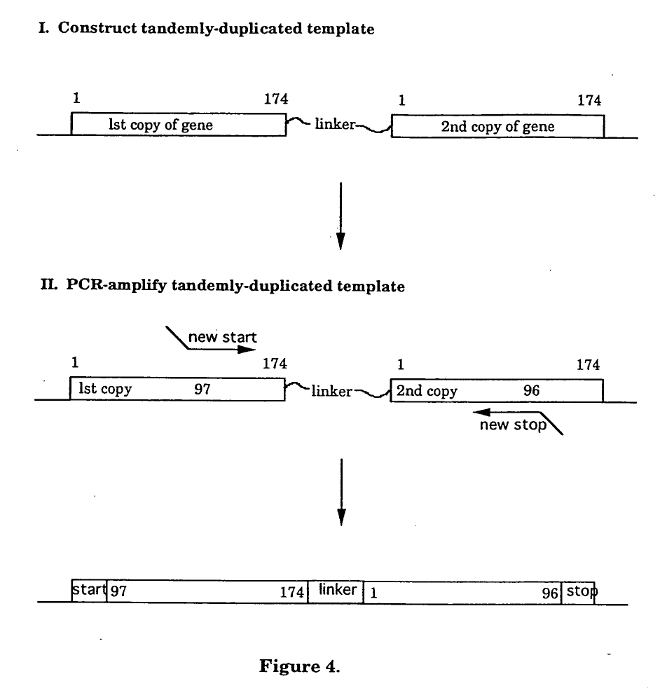

New N-terminus/C-terminus genes can be made based on the method described in R. A. Horlick, et al Protein Eng. 5:427-431 (1992). Polymerase chain reaction (PCR) amplification of the new N-terminus/C-terminus genes is performed using a tandemly duplicated template DNA. The steps are illustrated in FIG. 4.

The tandemly-duplicated template DNA is created by cloning and contains two copies of the gene separated by DNA sequence encoding a linker connecting the original C- and N-terminal ends of the two copies of the gene. Specific primer sets are used to create and amplify a full-length new N terminus/C-terminus gene from the tandemly-duplicated template DNA. These primers are designed to include appropriate restriction sites which allow for the cloning of the new gene into expression vectors. Typical PCR conditions are one cycle 95° C melting for two minutes; 25 cycles 94° C. denaturation for one minute, 50° C. annealing for one minute and 72° C. extension for one minute; plus one cycle 72° C. extension for seven minutes. A Perkin Elmer GeneAmp PCR Core Reagents kit (Perkin Elmer Corporation, Norwalk, Conn.) is used. A 100 ul reaction contains 100 pmole of each primer and one ug of template DNA; and 1× PCR buffer, 200 uM dGTP, 200 uM DATP, 200 uM dTTP, 200 uM dCTP, 2.5 units AmpliTaq DNA polymerase and 2 mM MgCl2. PCR reactions are performed in a Model 480 DNA thermal cycler (Perkin Elmer Corporation, Norwalk, Conn.). PCR reactions are purified using a Wizard PCR Preps kit (Promega).

### DNA Isolation and Characterization

Plasmid DNA can be isolated by a number of different methods and using commercially available kits known to those skilled in the art. A few such methods are shown herein. Plasmid DNA is isolated using the Promega Wizard™ Miniprep kit (Madison, Wis.), the Qiagen QIAwell Plasmid isolation kits (Chatsworth, Calif.) or Qiagen Plasmid Midi kit. These kits follow the same general procedure for plasmid DNA isolation. Briefly, cells are pelleted by centrifugation (5000×g), plasmid DNA released with sequential NaOH/acid treatment, and cellular debris is removed by centrifugation (10000×g). The supernatant (containing the plasmid DNA) is loaded onto a column containing a DNA-binding resin, the column is washed, and plasmid DNA eluted with TE. After screening for the colonies with the plasmid of interest, the E. Coli cells are inoculated into 50-100 mLs of LB plus appropriate antibiotic for overnight growth at 37° C. in an air incubator while shaking. The purified plasmid DNA is used for DNA sequencing, further restriction enzyme digestion, additional subcloning of DNA fragments and transfection into mammalian, E. coli or other cells.

### Sequence Confirmation

Purified plasmid DNA is resuspended in dH₂O and quantitated by measuring the absorbance at 260/280 nm in a Bausch and Lomb Spectronic 601 UV spectrometer. DNA samples are sequenced using ABI PRISM™ DyeDeoxy terminator sequencing chemistry (Applied Biosystems Division of Perkin Elmer Corporation, Lincoln City, Calif.) kits (Part Number 401388 or 402078) according to the manufacturers suggested protocol usually modified by the addition of 50 DMSO to the sequencing mixture. Sequencing reactions are performed in a Model 480 DNA thermal cycler (Perkin Elmer Corporation, Norwalk, Conn.) following the recommended amplification conditions. Samples are purified to remove excess dye terminators with Centri-Sep™ spin columns (Princeton Separations, Adelphia, N.J.) and lyophilized. Fluorescent dye labeled sequencing reactions are resuspended in deionized formamide, and sequenced on denaturing 4.75% polyacrylamide-8M urea gels using an ABI Model 373A automated DNA sequencer. Overlapping DNA sequence fragments are analyzed and assembled into master DNA contigs using Sequencher v2.1 DNA analysis software (Gene Codes Corporation, Ann Arbor, Mich.).

### Expression of EPO Receptor Agonists in Mammalian Cells Mammalian Cell Transfection/Production of Conditioned Media

The BHK-21 cell line can be obtained from the ATCC (Rockville, Md.). The cells are cultured in Dulbecco's modified Eagle media (DMEM/high-glucose), supplemented to 2 mM (mM) L-glutamine and 10% fetal bovine serum (FBS). This formulation is designated BHK growth media. Selective media is BHK growth media supplemented with 453 units/mL hygromycin B (Calbiochem, San Diego, Calif.). The BHK-21 cell line was previously stably transfected with the HSV transactivating protein VP16, which transactivates the IE110 promoter found on the plasmid pMON3359 (See Hippenmeyer et al., Bio/Technology, pp.1037-1041, 1993). The VP16 protein drives expression of genes inserted behind the IE110 promoter. BHK-21 cells expressing the transactivating protein VP16 are designated BHK-VP16. The plasmid pMON1118 (See Highkin et al., Poultry Sci., 70: 970-981, 1991) expresses the hygromycin resistance gene from the SV40 promoter. A similar plasmid is available from ATCC, pSV2-hph.

BHK-VP16 cells are seeded into a 60 millimeter (mm) tissue culture dish at 3×10⁵ cells per dish 24 hours prior to transfection. Cells are transfected for 16 hours in 3 mL of “OPTIMEM”™ (Gibco-BRL, Gaithersburg, Md.) containing 10 ug of plasmid DNA containing the gene of interest, 3 ug hygromycin resistance plasmid, pMON1118, and 80 ug of Gibco-BRL “LIPOFECTAMINE”™ per dish. The media is subsequently aspirated and replaced with 3 mL of growth media. At 48 hours post-transfection, media from each dish is collected and assayed for activity (transient conditioned media) . The cells are removed from the dish by trypsin-EDTA, diluted 1:10 and transferred to 100 mm tissue culture dishes containing 10 mL of selective media. After approximately 7 days in selective media, resistant cells grow into colonies several millimeters in diameter. The colonies are removed from the dish with filter paper (cut to approximately the same size as the colonies and soaked in trypsin/EDTA) and transferred to individual wells of a 24 well plate containing 1 mL of selective media. After the clones are grown to confluence, the conditioned media is re-assayed, and positive clones are expanded into growth media.

### Expression of EPO Receptor Agonists in E. coli

E. coli strain MON105 or JM101 harboring the plasmid of interest are grown at 37° C. in M9 plus casamino acids medium with shaking in a air incubator Model G25 from New Brunswick Scientific (Edison, N.J.). Growth is monitored at OD600 until it reaches a value of 1, at which time nalidixic acid (10 milligrams/mL) in 0.1 N NaOH is added to a final concentration of 50 μg/mL. The cultures are then shaken at 37° C. for three to four additional hours. A high degree of aeration is maintained throughout culture period in order to achieve maximal production of the desired gene product. The cells are examined under a light microscope for the presence of inclusion bodies (IB). One mL aliquots of the culture are removed for analysis of protein content by boiling the pelleted cells, treating them with reducing buffer and electrophoresis via SDS-PAGE (see Maniatis et al. Molecular Cloning: A Laboratory Manual, 1982). The culture is centrifuged (5000×g) to pellet the cells.

Additional strategies for achieving high-level expression of genes in E. coli can be found in Savwas, C. M. (Microbiological Reviews 60;512-538, 1996).

Inclusion Body preparation, Extraction, Refolding, Dialysis, DEAE Chromatography, and Characterization of the EPO receptor agonists which accumulate as inclusion bodies in E. coli.

### Isolation of Inclusion Bodies:

The cell pellet from a 330 mL E. coli culture is resuspended in 15 mL of sonication buffer (10 mM 2-amino-2-(hydroxymethyl) 1,3-propanediol hydrochloride (Tris-HCl), pH 8.0+1 mM ethylenediaminetetraacetic acid (EDTA)). These resuspended cells are sonicated using the microtip probe of a Sonicator Cell Disruptor (Model W-375, Heat Systems-Ultrasonics, Inc., Farmingdale, N.Y.). Three rounds of sonication in sonication buffer followed by centrifugation are employed to disrupt the cells and wash the inclusion bodies (IB). The first round of sonication is a 3 minute burst followed by a 1 minute burst, and the final two rounds of sonication are for 1 minute each.

### Extraction and Refolding of Proteins from Inclusion Body Pellets

Following the final centrifugation step, the IB pellet is resuspended in 10 mL of 50 mM Tris-HCl, pH 9.5, 8 M urea and 5 mM dithiothreitol (DTT) and stirred at room temperature for approximately 45 minutes to allow for denaturation of the expressed protein.

The extraction solution is transferred to a beaker containing 70 mL of 5mM Tris-HCl, pH 9.5 and 2.3 M urea and gently stirred while exposed to air at 4° C. for 18 to 48 hours to allow the proteins to refold. Refolding is monitored by analysis on a Vydac (Hesperia, Calif.) C18 reversed phase high pressure liquid chromatography (RP-HPLC) column (0.46×25 cm). A linear gradient of 40% to 65% acetonitrile, containing 0.1% trifluoroacetic acid (TFA), is employed to monitor the refold. This gradient is developed over 30 minutes at a flow rate of 1.5 mL per minute. Denatured proteins generally elute later in the gradient than the refolded proteins.

### Purification

Following the refold, contaminating E. coli Proteins are removed by acid precipitation. The pH of the refold solution is titrated to between pH 5.0 and pH 5.2 using 15% (v/v) acetic acid (HOAc). This solution is stirred at 4° C. for 2 hours and then centrifuged for 20 minutes at 12,000×g to pellet any insoluble protein.

The supernatant from the acid precipitation step is dialyzed using a Spectra/Por 3 membrane with a molecular weight cut off (MWCO) of 3,500 daltons. The dialysis is against 2 changes of 4 liters (a 50-fold excess) of lOmM Tris-HCl, pH 8.0 for a total of 18 hours. Dialysis lowers the sample conductivity and removes urea prior to DEAE chromatography. The sample is then centrifuged (20 minutes at 12,000×g) to pellet any insoluble protein following dialysis.

A Bio-Rad Bio-Scale DEAE2 column (7×52 mm) is used for ion exchange chromatography. The column is equilibrated in a buffer containing lOmM Tris-HCl, pH 8.0. The protein is eluted using a 0-to-500 mM sodium chloride (NaCl) gradient, in equilibration buffer, over 45 column volumes. A flow rate of 1 mL per minute is used throughout the run. Column fractions (2 mL per fraction) are collected across the gradient and analyzed by RP HPLC on a Vydac (Hesperia, Calif.) C18 column (0.46×25 cm). A linear gradient of 40% to 65% acetonitrile, containing 0.1% trifluoroacetic acid (TFA), is employed. This gradient is developed over 30 minutes at a flow rate of 1.5 mL per minute. Pooled fractions are then dialyzed against 2 changes of 4 liters (50-to-500-fold excess) of 10 mM ammonium acetate (NH₄Ac), pH 4.0 for a total of 18 hours. Dialysis is performed using a Spectra/Por 3 membrane with a MWCO of 3,500 daltons. Finally, the sample is sterile filtered using a 0.22 μm syringe filter (μStar LB syringe filter, Costar, Cambridge, Mass.), and stored at 4° C.

In some cases the folded proteins can be affinity purified using affinity reagents such as mAbs or receptor subunits attached to a suitable matrix. Alternatively, (or in addition) purification can be accomplished using any of a variety of chromatographic methods such as: ion exchange, gel filtration or hydrophobic chromatography or reversed phase HPLC.

These and other protein purification methods are described in detail in Methods in Enzymology, Volume 182 ‘Guide to Protein Purification’ edited by Murray Deutscher, Academic Press, San Diego, Calif. (1990).

### Protein Characterization

The purified protein is analyzed by RP-HPLC, electrospray mass spectrometry, and SDS-PAGE. The protein quantitation is done by amino acid composition, RP-HPLC, and Bradford protein determination. In some cases tryptic peptide mapping is performed in conjunction with electrospray mass spectrometry to confirm the identity of the protein.

### Methylcellulose Assay

This assay reflects the ability of colony stimulating factors to stimulate normal bone marrow cells to produce different types of hematopoietic colonies in vitro (Bradley et al., Aust. Exp Biol. Sci. 44:287-300, 1966), Pluznik et al., J. Cell Comp. Physio 66:319-324, 1965).

### Methods

Approximately 30 mL of fresh, normal, healthy bone marrow aspirate are obtained from individuals following informed consent. Under sterile conditions samples are diluted 1:5 with a 1× PBS (#14040.059 Life Technologies, Gaithersburg, Md.) solution in a 50 mL conical tube (#25339-50 Corning, Corning Md.). Ficoll (Histopaque 1077 Sigma H-8889) is layered under the diluted sample and centrifuged, 300×g for 30 min. The mononuclear cell band is removed and washed two times in 1× PBS and once with 1% BSA PBS (CellPro Co., Bothel, Wash.). Mononuclear cells are counted and CD34+ cells are selected using the Ceprate LC (CD34) Kit (CellPro Co., Bothel, Wash.) column. This fractionation is performed since all stem and progenitor cells within the bone marrow display CD34 surface antigen.

Cultures are set up in triplicate with a final volume of 1.0 mL in a 35×10 mm petri dish (Nunc#174926) . Culture medium is purchased from Terry Fox Labs. (HCC-4230 medium (Terry Fox Labs, Vancouver, B.C., Canada) and erythropoietin (Amgen, Thousand Oaks, Calif.) is added to the culture media. 3,000-10,000 CD34+ cells are added per dish. EPO receptor agonist proteins, in conditioned media from transfected mammalian cells or purified from conditioned media from transfected mammalian cells or E. coli, are added to give final concentrations ranging from 0.001 nM to 10 nM. Cultures are resuspended using a 3 cc syringe and 1.0 mL is dispensed per dish. Control (baseline response) cultures received no colony stimulating factors. Positive control cultures received conditioned media (PHA stimulated human cells: Terry Fox Lab. H2400). Cultures are incubated at 37° C., 5% CO₂ in humidified air.

Hematopoietic colonies which are defined as greater than 50 cells are counted on the day of peak response (days 10-11) using a Nikon inverted phase microscope with a 40× objective combination. Groups of cells containing fewer than 50 cells are referred to as clusters. Alternatively colonies can be identified by spreading the colonies on a slide and stained or they can be picked, resuspended and spun onto cytospin slides for staining.

### Human Cord Blood Hematopoietic Growth Factor Assays

Bone marrow cells are traditionally used for in vitro assays of hematopoietic colony stimulating factor (CSF) activity. However, human bone marrow is not always available, and there is considerable variability between donors. Umbilical cord blood is comparable to bone marrow as a source of hematopoietic stem cells and progenitors (Broxmeyer et al., PNAS USA 89:4109-113, 1992; Mayani et al., Blood 81:3252-3258, 1993). In contrast to bone marrow, cord blood is more readily available on a regular basis. There is also a potential to reduce assay variability by pooling cells obtained fresh from several donors, or to create a bank of cryopreserved cells for this purpose. By modifying the culture conditions, and/or analyzing for lineage specific markers, it is be possible to assay specifically for burst forming colonies (BFU-E) activity.

### Methods

Mononuclear cells (MNC) are isolated from cord blood within 24 hr. of collection, using a standard density gradient (1.077 g/mL Histopaque). Cord blood MNC have been further enriched for stem cells and progenitors by several procedures, including immunomagnetic selection for CD14−, CD34+ cells; panning for SBA−, CD34+ fraction using coated flasks from Applied Immune Science (Santa Clara, Calif.); and CD34+ selection using a CellPro (Bothell, Wash.) avidin column. Either freshly isolated or cryopreserved CD34+ cell enriched fractions are used for the assay. Duplicate cultures for each serial dilution of sample (concentration range from 1 pM to 1204 pM) are prepared with 1×104 cells in lml of 0.9% methylcellulose containing medium without additional growth factors (Methocult H4230 from Stem Cell Technologies, Vancouver, BC.). After culturing for 7-9 days, colonies containing >30 cells are counted.

### Transfected Cell Lines

Cell lines, such as BHK or the murine pro B cell line Baf/3, can be transfected with a colony stimulating factor receptor, such as the human EPO receptor which the cell line does not have. These transfected cell lines can be used to determine the cell proliferative activity and/or receptor binding.

### EXAMPLE 1

Genes encoding the sequence rearranged EPO ligands can be constructed by any one of the methods described herein or by other recombinant methods known to those skilled in the art. For the purpose of this example, the site of permutation is between residues 131(Arg) and 132(Thr) of EPO. This is a site which is susceptible to proteolytic cleavage, thereby indicating surface exposure with a relatively high degree of flexibility.

In this example a new N-terminus and a new C-terminus is created without a linker joining the original termini. This is done, as described in Method II, in 2 steps of PCR and a blunt end ligation.

In the first PCR step, using a vector containing the DNA sequence of SEQ ID NO:120 as the template, and the primers “new start” and “blunt start”, a DNA fragment is created which encodes the new N-terminus. This fragment is termed “fragment start”. The sequence underlined in the new start primer is the NcoI restriction site.

SEQ ID NO:131

| | |
|---|---|
| New start primer = | gcgcgcCCATGGACAATCACTGCTGAC |

SEQ ID NO:132

| | |
|---|---|
| Blunt start primer = | TCTGTCCCCTGTCCT |

In the second PCR step, using a vector containing the DNA sequence of SEQ ID NO:120 as the template, and the primers “new stop” and “blunt stop” create a DNA fragment which encodes the new C-terminus. This fragment is termed “fragment stop”. The sequence underlined in the new stop primer is the HindIII restriction site.

New stop primer =

SEQ ID NO:133

gcgcgcAAGCTTATTATCGGAGTCGAGCAGCTGAGGCCGCATC
Blunt end primer =

SEQ ID NO:134

GCCCCACCACGCCTCATCTGT

In the ligation step, the two fragments created in the two PCR reactions are ligated together, digested with NcoI and HindIII and cloned into an expression vector. The clones are screened by restiction analysis and DNA sequenced to confirm the proper sequence. The primers can be designed to create restriction sites other than NcoI and HindIII to clone into other expression vectors.

### EXAMPLE 2

The sequence rearranged EPO receptor agonists of the present invention can be assayed for bioactivity by the methods described herein or by other assays know to those skilled in the art.

Additional techniques for the construction of the variant genes, recombinant protein expression , protein purification, protein characterization, biological activity determination can be found in WO 94/12639, WO 94/12638, WO 95/20976, WO 95/21197, WO 95/20977, WO 95/21254 which are hereby incorporated by reference in their entirety.

All references, patents or applications cited herein are incorporated by reference in their entirety as if written herein.

Various other examples will be apparent to the person skilled in the art after reading the present disclosure without departing from the spirit and scope of the invention. It is intended that all such other examples be included within the scope of the appended claims.

We claim:

1. A human eythropoietin receptor agonist polypeptide, comprising a modified erythropoietin amino acid sequence selected from the group consisting of:
(a) the sequence of SEQ ID No:121;
(b) a polypeptide sequence comprising residues 7-166 of SEQ ID NO:121;
(c) a polypeptide sequence comprising residues 1-161 of SEQ ID NO:121; and
(d) a polypeptide sequence comprising residues 7-161 of SEQ ID NO:121;
and wherein said modification comprises the linear rearrangement of the sequences of (a)-(d) wherein the N-terminus is joined to the C-terminus directly or through a linker capable of joining the N-terminus to the C-terminus and new C- and N-termini are created between the amino acid residue pairs of SEQ ID NO:121 selected from the group consisting of:
23-24
28-29
33-34
34-35
35-36
36-37
37-38
38-39
40-41
41-42
43-44
44-45
45-46
46-47
47-48
48-49
50-51
51-52
52-53
53-54
54-55
55-56
56-57
57-58
77-78
78-79
84-85
85-86
86-87
108-109
109-110
110-111
111-112
112-113
113-114
114-115
117-118
118-119
119-120
122-123
123-124
124-125
125-126
126-127
127-128
128-129
129-130
130-131 and
131-132;
and wherein said erythropoietin receptor agonist polypeptide may optionally be immediately preceded by (methionine⁻¹) (alanine⁻¹) or (methionine 2⁻², alanine⁻¹).

2. The erythropoietin receptor agonist polypeptide, as recited in claim 1, wherein said linker is selected from the group consisting of:

| | |
|---|---|
| GlyGlyGlySer; | SEQ ID NO:123 |
| GlyGlyGlySerGlyGlyGlySer; | SEQ ID NO:124 |
| GlyGlyGlySerGlyGlyGlySerGlyGlyGlySer; | SEQ ID NO:125 |
| SerGlyGlySerGlyGlySer; | SEQ ID NO:126 |
| GluPheGlyAsnMet; | SEQ ID NO:127 |
| GluPheGlyGlyAsnMet; | SEQ ID NO:128 |
| GluPheGlyGlyAsnGlyGlyAsnMet; | SEQ ID NO:129 |
| and | |
| GlyGlySerAspMetAlaGly. | SEQ ID NO:130 |

3. The erythropoietin receptor agonist polypeptide of claim 1 selected from the group consisting of:
SEQ ID NO:1; SEQ ID NO:6; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:31; SEQ ID NO:32; SEQ ID NO:33; SEQ ID NO:36; SEQ ID NO:37; SEQ ID NO:38; SEQ ID NO:39; SEQ ID NO:40; SEQ ID NO:41; SEQ ID NO:42; SEQ ID NO:45; SEQ ID NO:46; SEQ ID NO:47; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59 and SEQ ID NO:122.

4. The erythropoietin receptor agonist polypeptide of claim 3 wherein the linker sequence is selected from the group consisting of:

| | |
|---|---|
| GlyGlyGlySerGlyGlyGlySer; | SEQ ID NO:124 |
| GlyGlyGlySerGlyGlyGlySerGlyGlyGlySer; | SEQ ID NO:125 |
| SerGlyGlySerGlyGlySer; | SEQ ID NO:126 |
| GluPheGlyAsnMet; | SEQ ID NO:127 |
| GluPheGlyGlyAsnMet; | SEQ ID NO:128 |
| GluPheGlyGlyAsnGlyGlyAsnMet; | SEQ ID NO:129 |
| and | |
| GlyGlySerAspMetAlaGly. | SEQ ID NO:130 |

5. A nucleic acid molecule comprising a DNA sequence encoding the erythropoietin receptor agonist polypeptide of claim 1.

6. A nucleic acid molecule comprising a DNA sequence encoding the erythropoietin receptor agonist polypeptide of claim 2.

7. A nucleic acid molecule comprising a DNA sequence encoding the erythropoietin receptor agonist polypeptide of claim 3.

8. A nucleic acid molecule comprising a DNA sequence encoding the erythropoietin receptor agonist polypeptide of claim 3 selected from the group consisting of:
SEQ ID NO:60; SEQ ID NO:65; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84; SEQ ID NO:90; SEQ ID NO:91; SEQ ID NO:92; SEQ ID NO:95; SEQ ID NO:96; SEQ ID NO:97; SEQ ID NO:98; SEQ ID NO:99; SEQ ID NO:100; SEQ ID NO:101; SEQ ID NO:104; SEQ ID NO:105; SEQ ID NO:106; SEQ ID NO:109; SEQ ID NO:110; SEQ ID NO:111; SEQ ID NO:112; SEQ ID NO:113; SEQ ID NO:114; SEQ ID NO:115; SEQ ID NO:116; SEQ ID
NO:117; SEQ ID NO:118 and SEQ ID NO:119.

9. A nucleic acid molecule comprising a DNA sequence encoding the erythropoietin receptor agonist polypeptide of claim 4.

10. A method of producing a erythropoietin receptor agonist polypeptide comprising: growing under suitable nutrient conditions, a host cell transformed or transfected with a replicable vector comprising said nucleic acid molecule of claim 5, 6, 7, 8 or 9 in a manner allowing expression of said erythropoietin receptor agonist polypeptide and recovering said erythropoietin receptor agonist polypeptide.

11. A composition comprising a erythropoietin receptor agonist polypeptide according to claim 1, 2, 3 or 4; and a pharmaceutically acceptable carrier.

12. A composition comprising a erythropoietin receptor agonist polypeptide according to claim 1, 2, 3 or 4; a second protein; and a pharmaceutically acceptable carrier.

13. The composition of claim 12 wherein said second protein is selected from the group consisting of: GM-CSF, G-CSF, c-mpl ligand, M-CSF, IL-1, IL-4, IL-2, IL-3, IL-5, IL-6, IL-7, IL-8, IL-9, IL-10, IL-11, IL-12, IL-13, IL-15, LIF, flt3/flk2 ligand, human growth hormone, B-cell growth factor, B-cell differentiation factor, eosinophil differentiation factor, stem cell factor, IL-3 variant, fusion protein, G-CSF receptor agonist, c-mpl receptor agonist, IL-3 receptor agonist, and multi-functional receptor agonist.

14. A method of stimulating the production of hematopoietic cells in a patient comprising the step of; administering a erythropoietin receptor agonist polypeptide of claim 1, 2, 3 or 4, to said patient.
